(19) United States
(12) Patent Application Publication
(10) Pub. No.: US 2006/0281948 A1
(43) Pub. Date: Dec. 14, 2006

(54) Process for preparing substituted benzoyl cyanides

(75) Inventors: Andreas Job, Koln (DE); Bjorn Schlummer, Bonn (DE)

Correspondence Address: LANXESS CORPORATION, 111 RIDC PARK WEST DRIVE, PITTSBURGH, PA, 15275-1112, US

(73) Assignee: Saltigo GmbH

(21) Appl. No.: 11/450,066

(22) Filed: Jun. 9, 2006

(30) Foreign Application Priority Data
Jun. 11, 2005 (DE) 10 2005 027 149.9

(51) Int. Cl. C07C 255/17

(52) U.S. Cl. 562/869

(57) ABSTRACT

Process for preparing substituted benzoyl cyanides of the general formula (I) 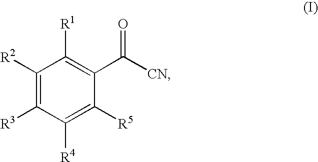
where R¹, R², R³, R⁴, R⁵ are each independently hydrogen, chlorine, bromine, iodine, fluorine, a C1-C8-alkyl, aryl, arylalkyl, C1-C8-alkoxy or C1-C8-alkylmercapto radical or are —CN, —COOR⁶, —CONR₂⁷, —SO₃R⁸ or —SO₂NR₂⁹, where R⁶, R⁷, R⁸, R⁹ are each independently a C1-C8-alkyl radical, by
a) reacting benzoyl chlorides of the general formula (II) 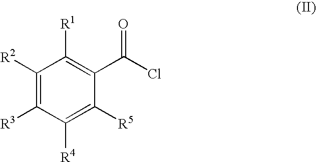 in which
R¹, R², R³, R⁴, R⁵ are each as defined in formula I, with 0.9-1.4 molar equivalents of copper cyanide without further solvent, optionally under elevated pressure, under inert gas atmosphere at a reaction temperature between 150 and 165° C., b) after a reaction time of not more than 5 hours and cooling to a temperature below 100° C., an aprotic organic solvent being added to the reaction mixture to precipitate out the copper salt formed and c) after the copper salt precipitated has been filtered off, the remaining filtrate being cooled to a temperature between −40 and +20° C. to crystallize out the crude product and d) the crude product crystallized out being removed from the aprotic organic solvent and optionally dried.

The present invention relates to an improved process for preparing substituted, preferably halogen-substituted, benzoyl cyanides.

An overview of the preparation of benzoyl cyanides is given in Angew. Chem., Int. Ed. Engl. 1982, 21, 36, Scheme 1. This describes two general routes starting from benzoyl chlorides. First, benzoyl cyanides can be obtained from the corresponding chlorides by reacting them, for example, with trimethylsilyl cyanide according to Synthesis 1979, 204. Additionally described is the reaction of benzoyl chlorides with heavy metal cyanides, which, however, is described as disadvantageous in the respect that the reaction mixture has to be heated to temperatures of 120-160° C. over periods of hours, which leads to the suspicion that the formation of secondary components is promoted. In contrast, a more advantageous process described therein is that in which benzoyl chlorides can be reacted with copper cyanide with addition of acetonitrile even at 80° C. with a yield of up to 75%. However, the removal of the copper cyanide used as a reagent and of copper chloride formed in the reaction by means of simple filtration appears to be difficult to those skilled in the art, since these salts are known to form soluble complexes with precisely the acetonitrile used. With a rather exotic and industrially unavailable cyanide, thallium cyanide, yields of benzoyl cyanides of 89% are achieved even at 20° C. according to the aforementioned article.

DE-A 3 035 021, DE-A 4 311 722 and EP-A 1 316 613 disclose reactions of benzoyl chlorides with copper cyanide with addition of acetonitrile, in which the resulting crude products, after separation, are removed from the copper salts by filtration and subsequently optionally, after washing with water, distilled for purification. This procedure would be highly uneconomic for an industrial-scale reaction and additionally applicable only to substances which can be distilled under industrial conditions.

DE-A 2 624 891, DE-A 2 708 182 and U.S. Pat. No. 4,209,462 disclose preparation processes of benzoyl cyanides from benzoyl chlorides using superstoichiometric amounts of sodium cyanide and substoichiometric amounts of copper, copper oxide or copper cyanide and optionally additions of acetonitrile and optionally further solvents. It is common to all of these procedures that the resulting crude products have to be distilled in a separate step for purification. A disadvantage of this procedure is the restriction to distillable benzoyl cyanides and also, in the case of DE-A 2 624 891 and DE-A 2 708 182, the restricted recyclability of the waste streams, since they consist of solvent mixtures in the examples adduced.

US-2003158435 discloses a process for preparing benzoyl cyanide from benzoyl chloride in an aqueous biphasic system. In this process, sodium cyanide, sodium iodide and sodium hydroxide solution are used as reagents. This procedure is disadvantageous in that it can be utilized only to prepare water-stable compounds.

J. Fluorine Chem. 1993, 61, 117 describes the reaction of benzoyl chlorides with 1.5 equivalents of copper cyanide and catalytic amounts of phosphorus pentoxide in toluene, the reaction mixture being heated to reflux temperature over 40 hours. This method is disadvantageous as a result of the use of an excess of 0.5 equivalent of cyanide reagent, the addition of reactive phosphorus pentoxide and owing to the high thermal stress, since a multitude of side reactions can be suspected in this case. This is confirmed by the need to purify the resulting crude products in a very complicated manner by a filtration, subsequent washing with water, chromatographic purification and also a recrystallization. The resulting yields for the examples described (5 examples) are, as expected, low at 45-76%.

GB-A 2 395 483 describes the preparation of 2,3-dichlorobenzoyl cyanide in such a way that 2,3-dichlorobenzoyl chloride is reacted with 1.4 equivalents of copper cyanide at 160-165° C. for 6 hours. The workup includes firstly dilution with toluene, a filtration and, after the toluene has been distilled off, recrystallization of the crude product. A disadvantage of this preparation process is the long thermal stress both by the reaction and by the distillation of the toluene added, which likewise requires several hours on the industrial scale. The purity of the product is reported as 97% after gas chromatography analysis, but the typical secondary components in this reaction, such as the corresponding anhydride or the acid, typically cannot be detected efficiently by means of gas chromatography. The reproduction of this reaction has led to a distinct secondary component fraction in the product.

It is therefore an object of the invention to develop a process for the economically viable preparation of substituted, especially halogen-substituted, benzoyl cyanides in a good purity.

process has now been found for preparing substituted benzoyl cyanides of the general formula I 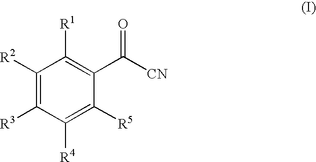
where R¹, R², R³, R⁴, R⁵ are each independently hydrogen, chlorine, bromine, iodine, fluorine, a C1-C8-alkyl, aryl, arylalkyl, C1-C8-alkoxy or C1-C8-alkylmercapto radical or are —CN, —COOR⁶, —CONR₂⁷, —SO₃R⁸ or —SO₂NR₂⁹, where R⁶, R⁷, R⁸, R⁹ are each independently a C1-C8-alkyl radical, by reacting benzoyl chlorides of the general formula II 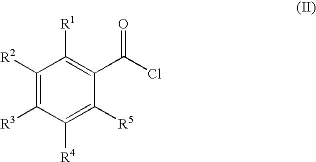
in which
R¹, R², R³, R⁴, R⁵ are each as defined in formula I,
   with copper cyanide, in which the reaction proceeds in substance, i.e. in the absence of organic solvents, and also other additives, the workup being effected by using at most one inert organic solvent or a solvent mixture (optionally “distillation cut” in customary technical-grade quality) and the product, after removal of the inorganic by-products by filtration, being isolated by crystallization out of the solvent.

The resulting product has a purity of at least 96% based on all ingredients.

The invention therefore provides a process for preparing halogen-substituted benzoyl cyanides of the general formula (I) 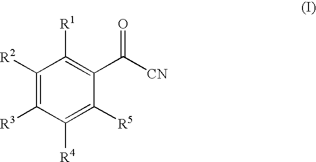
where R¹, R², R³, R⁴, R⁵ are each independently hydrogen, chlorine, bromine, iodine, fluorine, a C1-C8-alkyl, aryl, arylalkyl, C1-C8-alkoxy or C1-C8-alkylmercapto radical or are —CN, —COOR⁶, —CONR₂⁷, —SO₃R⁸ or —SO₂NR₂⁹, where R⁶, R⁷, R⁸, R⁹ are each independently a C1-C8-alkyl radical, by
a) reacting benzoyl chlorides of the general formula (II) 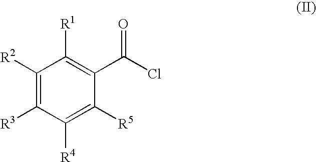
in which
   R¹, R², R³, R⁴, R⁵ are each as defined in formula I,
   with 0.9-1.4 molar equivalents of copper cyanide without further solvent, optionally under elevated pressure, under inert gas atmosphere at a reaction temperature between 150 and 165° C.,
   b) after a reaction time of not more than 5 hours and cooling to a temperature below 100° C., an aprotic organic solvent being added to the reaction mixture to precipitate out the copper salt formed and
   c) after the copper salt precipitated has been filtered off, the remaining filtrate being cooled to a temperature between −40 and +20° C. to crystallize out the crude product and
   d) the crude product crystallized out being removed from the aprotic organic solvent and optionally dried.

In the process according to the invention, the substituted benzoyl chloride is reacted with copper cyanide in substance for not more than 5 hours, preferably for not more than 4 hours, at a temperature in the range of 150 to 165° C, preferably 155-160° C. Comparative experiments, especially according to GB-A-2395483, have shown that the formation of secondary components increases greatly with increasing reaction time, which has an adverse effect on the resulting product quality. Surprisingly, it is possible in accordance with the invention, while maintaining the reaction times, to use a distinctly lower amount of cyanating reagent of approx. 1.1 equivalent instead of the 1.4 equivalents described in GB 2395483 or the 1.5 equivalents described in J. Fluorine Chem. 1993, 61, 117, without observing losses in the product yield. Since the reagents are very poisonous substances, this inventive procedure brings about a distinctly lower level of environmental pollution as a result of a distinctly reduced level of disposal complexity.

Typically, 0.9 to 1.4 molar equivalents (based on the benzoyl chloride), preferably from 1.0 to 1.3 molar equivalents, more preferably 1.0-1.1 molar equivalents of copper cyanide are used.

Since reactants and products are water-sensitive, operation is effected under inert gas atmosphere. The inert gas used is nitrogen or noble gases such as helium or argon.

The substituted benzoyl chlorides prepared in accordance with the invention may be those in which R1, R2, R3, R4 and R5 are each independently as described in formula I. Preferred C1-C8-alkyl radicals are methyl, ethyl, propyl and butyl radicals, preferred aryl radicals are phenyl or substituted phenyl radicals, and preferred arylalkyl radicals are benzyl or substituted benzyl radicals.

The process according to the invention is particularly suitable for preparing halogen-substituted benzoyl cyanides, i.e. at least one of the R1, R2, R3, R4 or R5 radicals therein is chlorine, bromine, iodine or fluorine. Preferred compounds are the 2,3-, 2,4-, 2,5- or 2,6- or 3,4-dihalobenzoyl cyanides, especially 2,3-dichlorobenzoyl cyanide, 2,4-dichlorobenzoyl cyanide, 2,5-dichlorobenzoyl cyanide, 2,6-dichlorobenzoyl cyanide, 3,4-dichlorobenzoyl cyanide and 4-bromo-2-chlorobenzoyl cyanide and 4-bromo-2-fluorobenzoyl cyanide, the 2-, 3- and 4-monohalobenzoyl cyanides, especially 4-bromobenzoyl cyanide and 4-fluorobenzoyl cyanide, the 2,3,4-, 2,3,5-, 2,4,6-, 2,3,6-trihalobenzoyl cyanides, especially 2,3,4-trichlorobenzoyl cyanide and 4-bromo-2,6-dichlorobenzoyl cyanide.

Surprisingly, the inorganic copper salts can be removed by diluting the reaction mixture directly with the solvent or solvent mixture suitable for crystallization and, after filtration from the copper salts, by crystallization of the product from this solution. As a result of this procedure, further steps which, as a result of a prolonged residence time or simultaneous further thermal stress on the crude product, as described, for example, in GB-A-2395483, in the course of the distillation of the toluene used to remove the copper salt formed, or described in J. Fluorine Chem. 1993, 61, 117, where water washings are carried out beforehand in addition to toluene distillation, favour the formation of secondary components and therefore markedly adversely affect the product quality are dispensed with. In addition to the improved product quality, the saving of at least one solvent which is typically used in distinctly larger amounts than the product itself is achieved. The associated saving of raw materials and the removal thereof constitute an active effect for improving the environmental friendliness of the process, and also an associated crucially improved economic viability.

The inert aprotic organic solvent used to remove the copper salts and also for crystallization belongs typically to the group of the nonpolar aliphatic C₂-C₈-alkanes, for example n-hexane and its isomers, n-heptane and its isomers, n-octane and its isomers, and also cycloalkanes such as cyclopentane, cyclohexane, cycloheptane, cyclooctane, and also cycloalkanes substituted with alkyl groups such as methyl, ethyl, propyl, isopropyl, n-butyl, sec-butyl and tert-butyl. It is equally possible to use mixtures of the aforementioned solvents, in particular industrially available distillation cuts such as technical-grade hexane, petroleum ether 50-70, 60-80, 70-90, 90-110, and also carbon dioxide when operation is effected under pressure. It is also equally possible to use nonpolar fluorinated solvents, for example fluorinated C₂-C₈-alkanes or alkenes such as perfluoroethylene, or fluorinated aromatic compounds such as hexafluorobenzene and octafluorotoluene. 4-Chlorobenzotrichloride is also possible. Preference is given to using (technical-grade) hexane as the solvent in step b).

The aprotic solvents used as the solvent typically have a water content of not more than 0.5% by weight, preferably of not more than 0.2% by weight, more preferably of not more than 0.1% by weight and in particular of not more than 0.05% by weight. Aprotic hydrocarbons with this water content are commercially available as solvents, but may also be dried before the reaction by appropriate incipient distillation.

The copper salts can also be removed under pressure, in which case, owing to the elevated pressure and thus also the elevated boiling point or boiling range of the solvent or of the solvent mixture, a higher isolation temperature is possible and thus, if appropriate, a higher concentration of crude product in the particular solvent or solvent mixture can be achieved.

For this purpose, it is likewise possible, in addition to the inert organic solvents which have been mentioned above, also to use alkanes such as ethane, propane, butane, and also carbon dioxide, under pressure. Under elevated pressure, it is possible with these solvents in the so-called supercritical state to remove the product from the copper salts, the product in this case being obtained by pressure reduction and simultaneous evaporation of the solvent.

For crystallization, the solution of the crude product in the inert organic solvent is cooled from its boiling point or just below it to a temperature of −40 to +20° C., preferably −20 to +10° C., more preferably −10 to +5° C. (The cooling can be effected either by jacket cooling or by reduced-pressure evaporative cooling.)

An essential feature of the process according to the invention is the particularly high purity with which the products are obtained. The inventive procedure can afford substituted benzoyl cyanides with a purity of >94%, preferably of >95%, more preferably of >96%, and the customary by-products of this reaction, such as the correspondingly substituted benzoic acid and the correspondingly substituted benzoic anhydride, are obtained with a content of <2%, preferably of <1%, more preferably of <0.6%, or with a content of <6%, preferably of <4%, more preferably of <3%.

The economic viability of the process can be increased further by distilling the solvent out of the mother liquor and using it again.

The copper salts filtered off may likewise be sent to copper recovery according to the known prior art, since they are obtained in relatively clean form.

### EXAMPLES

All reactions were carried out under nitrogen atmosphere. Unless stated otherwise, the analysis of the resulting products was carried out by means of HPLC analysis in order to ensure that the customary by-products such as the corresponding benzoic acids and also their anhydrides are detected.

### Comparative Example 1 (According to GB-A-2395483)

In a flask, 106.9 g (0.5 mol) of 2,3-dichlorobenzoyl chloride were melted at approx. 35-40° C. At this temperature, 62.8 g (0.7 mol) of copper cyanide were added with stirring.

This mixture was heated to 165° C. for 6 hours, in the course of which the grey suspension changed colour to brown-green. The reaction mixture was subsequently cooled to approx. 80° C. At this temperature, 50 ml of toluene were added and the suspension was hot-filtered through a sintered glass suction filter (G3 pore). The reaction flask and also the residue in the suction filter were each flushed three times with 20 ml of toluene each time. 79.0 g of moist solid remained on the suction filter.

The filtrate collected was concentrated at a temperature of 50° C. and a pressure of 25 hpa to a residue of 105.3 g. This was suspended in 650 ml of hexane at boiling temperature (69° C.), and insoluble fractions were left behind. The suspension was clarified by filtration at approx. 60° C. to leave 21.4 g of a moist crumbly solid. Analysis of the residue showed a content of >60% of 2,3-dichlorobenzoic anhydride. The content of 2,3-dichlorobenzoic acid was not determined.

The filtrate heated to approx. 60° C. was then cooled down to 15° C., in the course of which the product precipitated out in the form of yellow crystals with brownish oil fractions. It was observed that a portion of the product (especially the oily fractions) adhered on the glass wall of the flask.

After the adhered substance had been removed with a spatula, the suspension was filtered and the solid thus obtained was dried at a temperature of 40° C. and a pressure of 20 hPa. 63.8 g of 2,3-dichlorobenzoyl cyanide were obtained in the form of yellow crystals with oil fractions. The purity of the product was 89.4%. This corresponded to a yield of 57% of theory.

As a secondary component in the product, 6.9% of the corresponding 2,3-dichlorobenzoic anhydride were also found. The content of 2,3-dichlorobenzoic acid was not determined.

### Comparative Example 2 (According to GB-A-2395483)

In a flask, 256.5 g (1.2 mol) of 2,3-dichlorobenzoyl chloride were melted at approx. 35-40° C. At this temperature, 150.8 g (1.7 mol) of copper cyanide were added with stirring.

This mixture was heated to 165° C. for 6 hours, in the course of which the grey suspension changed colour to brown-green. The reaction mixture was subsequently cooled to approx. 80° C. At this temperature, 754 ml of toluene were added and the mixture was cooled further to 65° C. At this temperature, a gentle nitrogen stream was passed through the solution for 3 hours. Subsequently, the suspension was hot-filtered through a sintered glass suction filter (G3 pore). The filtration residue in the suction filter was flushed twice with 100 ml of toluene each time and then disposed of.

The filtrate collected was concentrated at a temperature of 50° C. and a pressure of 25 hPa to a residue of 259.4 g. This was suspended in 1670 ml of hexane at boiling temperature (69° C.), and insoluble fractions were left behind. The suspension was clarified by filtration at approx. 60° C. to leave 16.7 g of a moist crumbly solid. Analysis of the residue showed a content of >60% of 2,3-dichlorobenzoic anhydride. The content of 2,3-dichlorobenzoic acid was not determined.

The filtrate heated to approx. 60° C. was then cooled down to 15° C., in the course of which the product precipitated out in the form of yellow crystals with brownish oil fractions. It was observed that a portion of the product (especially the oily fractions) adhered on the glass wall of the flask.

After the adhered substance had been removed with a spatula, the suspension was filtered and the solid thus obtained was dried at a temperature of 40° C. and a pressure of 20 hPa. 185 g of 2,3-dichlorobenzoyl cyanide were obtained in the form of yellow crystals with oil fractions. The purity of the product was 84%. This corresponded to a yield of 66% of theory.

As a secondary component in the product, 9.8% of the corresponding 2,3-dichlorobenzoic anhydride were also found. The content of acid was not determined.

### Comparative Example 3 (Based on DE-A-2708182/DE-A-2624891)

In a flask, 53.4 g (0.25 mol) of 2,3-dichlorobenzoyl chloride were melted at approx. 35-40° C. At this temperature, 14.7 g (0.3 mol) of sodium cyanide and 2.2 g (0.03 mol) of copper cyanide were added with stirring.

This mixture was heated to 165° C. for 6 hours. The analysis of the crude mixture showed the following result:

51.8% 2,3-dichlorobenzoyl chloride, 38.5% 2,3-dichlorobenzoyl cyanide and 9.0% 2,3-dichloro-benzoic anhydride. The content of 2,3-dichlorobenzoic acid was not determined.

The reaction mixture was subsequently stirred at 165° C. for a further 4 hours and cooled to approx. 80° C. At this temperature, 157 ml of toluene were added and the suspension was hot-filtered through a sintered glass suction filter (G3 pore). The filtration residue in the suction filter was flushed twice with 20 ml of toluene each time and subsequently disposed of.

The collected filtrate was concentrated to a residue of 54.2 g at a temperature of 50° C. and a pressure of 10 hPa. An analysis of the remaining dark oil showed the following result:

24.2% 2,3-dichlorobenzoyl chloride, 54.6% 2,3-dichlorobenzoyl cyanide and 5.0% 2,3-dichloro-benzoic anhydride. The content of 2,3-dichlorobenzoic acid was not determined.

### Example 1 (Inventive)

A flask was initially charged with 251.4 g (2.8 mol) of copper cyanide. After the flask had been heated to 45° C., 427.5 g (2.0 mol) of molten 2,3-dichlorobenzoyl chloride were added with stirring.

This mixture was heated to 160° C. for 4 hours, in the course of which the grey suspension changed colour to grey-green. The reaction mixture was subsequently cooled to approx. 60° C. At this temperature, 2765 ml of (technical-grade) hexane were added and the suspension was hot-filtered through a sintered glass suction filter (G3 pore). The reaction flask, and also the residue in the suction filter, were washed twice with 240 ml of (technical-grade) hexane each time. A grey solid remained on the suction filter: dry weight: 267.9 g, which was disposed of.

The collected filtrate was heated to boiling in a second flask for 30 min (approx. 69° C.) and subsequently cooled to 0° C. In the course of this, the product crystallized out in the form of yellow crystals with a very small amount of oily fractions. In contrast to the observation in Example 1, only a negligible film was present on the edge of the flask.

This suspension was filtered and the solid thus obtained was dried at a temperature of 30° C. and a pressure of 8 mbar. 345.8 g of 2,3-dichlorobenzoyl cyanide were obtained in the form of yellow crystals. The purity of the product was 97.4%. This corresponded to a yield of 84% of theory.

### Example 2 (Inventive)

In a flask, 106.9 g (0.5 mol) of 2,3-dichlorobenzoyl chloride were melted at approx. 35-40° C. At this temperature, 49.3 g (0.55 mol) of copper cyanide were added with stirring.

This mixture was heated to 165° C. for 4 hours, in the course of which the grey suspension changed colour to light brown. The reaction mixture was subsequently cooled to approx. 60° C. At this temperature, 691 ml of hexane were added and the suspension was hot-filtered. The filter residue, 81.6 g of moist solid, was not washed again and was discarded.

The filtrate heated to approx 60° C. was then cooled down to 15° C. In the course of this, the product crystallized out in the form of yellow crystals with a very small amount of oily fractions. In contrast to the observation in Example 1, only a negligible film was present on the edge of the flask.

The suspension was filtered through a reverse sintered glass suction filter and blown dry with nitrogen for one hour. 76.8 g of 2,3-dichlorobenzoyl cyanide were obtained in the form of yellow crystals. The purity of the product was 97.5%. The crystals exhibited a drying loss of <0.2%. This corresponded to a yield of 74% of theory.

As a secondary component in the product, 1.8% of the corresponding 2,3-dichlorobenzoic anhydride and 0.5% 2,3-dichlorobenzoic acid (remove if appropriate) were also found.

### Example 3 (Inventive)

A flask was initially charged with 197.0 g (2.2 mol) of copper cyanide. After the flask had been heated to 45° C, 427.5 g (2.0 mol) of molten 2,3-dichlorobenzoyl chloride were added with stirring.

This mixture was heated to 160° C. for 4 hours, in the course of which the grey suspension changed colour to grey-green. The reaction mixture was subsequently cooled to approx. 60° C. At this temperature, 2765 ml of (technical-grade) hexane were added and the suspension was hot-filtered through a sintered glass suction filter (G3 pore). The reaction flask, and also the residue in the suction filter, were washed twice with 240 ml of (technical-grade) hexane each time. A grey solid remained on the suction filter: dry weight: 212.3 g, which was disposed of.

The collected filtrate was heated to boiling (approx. 69° C.) in a second flask for 30 min and subsequently cooled to 0° C. In the course of this, the product crystallized out in the form of yellow crystals with a very small amount of oily fractions. In contrast to the observation in Example 1, only a negligible film was present on the edge of the flask.

This suspension was filtered and the solid thus obtained was dried at a temperature of 30° C. and a pressure of 8 mbar. 344.6 g of 2,3-dichlorobenzoyl cyanide were obtained in the form of yellow crystals. The purity of the product was 98.0%. This corresponded to a yield of 84% of theory.

### Example 4 (Analysis Comparison of GC vs HPLC)

An analysis comparison of a sample of 2,3-dichlorobenzoyl cyanide (prepared by Comparative Example 1) gave the following result (two determinations in each case were carried out):

| | Content of | Content of | Content of | Content of |
|---|---|---|---|---|
| | 2,3- | 2,3- | 2,3- | 2,3- |
| | dichloro- | dichloro- | dichloro- | dichloro- |
| Analysis | benzoyl | benzoyl | benzoic | benzoic |
| type | chloride | cyanide | acid | anhydride |
| GC | 0.00 | 99.34 | 0.00 | 0.61 |
| (1st det) | | | | |
| GC | 0.00 | 99.32 | 0.00 | 0.62 |
| (2nd det.) | | | | |
| HPLC | 0.05 | 92.61 | 1.69 | 5.52 |
| (1st det.) | | | | |
| HPLC | 0.06 | 92.82 | 1.58 | 5.37 |
| (2nd det.) | | | | |

This table makes clear that not all secondary components of the reaction or in some cases too low a secondary component content are indicated by means of gas chromatography analysis. Reliable results are obtained by means of HPLC analysis.

1. Process for preparing substituted benzoyl cyanides of the general formula (I) 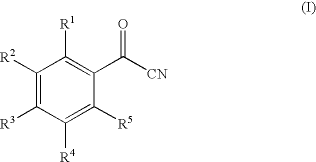
where R¹, R², R³, R⁴, R⁵ are each independently hydrogen, chlorine, bromine, iodine, fluorine, a C1-C8-alkyl, aryl, arylalkyl, C1-C8-alkoxy or C1-C8-alkylmercapto radical or are —CN, —COOR⁶, —CONR₂⁷, —SO₃R⁸ or —SO₂NR₂⁹, where R⁶, R⁷, R⁸, R⁹ are each independently a C1-C8-alkyl radical, by
a) reacting benzoyl chlorides of the general formula (II) 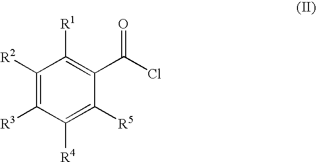
in which
R¹, R², R³, R⁴, R⁵ are each as defined in formula I
with 0.9-1.4 molar equivalents of copper cyanide without further solvent, optionally under elevated pressure, under inert gas atmosphere at a reaction temperature between 150 and 165° C.,
b) after a reaction time of not more than 5 hours and cooling to a temperature below 100° C., an aprotic organic solvent being added to the reaction mixture to precipitate out the copper salt formed and
c) after the copper salt precipitated has been filtered off, the remaining filtrate being cooled to a temperature between −40 and +20° C. to crystallize out the crude product and
d) the crude product crystallized out being removed from the aprotic organic solvent and optionally dried.

2. Process according to claim 1, wherein the aprotic organic solvent is a C₂-C₈-alkane, an optionally substituted cycloalkane or mixtures thereof, a fluorinated C₂-C₈-alkane or alkene, a fluorinated aromatic or carbon dioxide.

3. Process according to claim 1, wherein the aprotic organic solvent is hexane or a technical-grade hexane mixture.

4. Process according to claim 1, wherein the aprotic organic solvent is a technical-grade distillation cut such as 50-70 petroleum ether, 60-80 petroleum ether, 70-90 petroleum ether or 90-110 petroleum ether.

5. Process according to claims 1, wherein the substituted benzoyl cyanide is a halogen-substituted benzoyl cyanide in which at least one of the R1, R2, R3, R4 or R5 radicals is chlorine, fluorine, bromine or iodine.

6. Process according to claim 5, wherein the halogen-substituted benzoyl cyanide is 2,3-dichlorobenzoyl cyanide, 2,4-dichlorobenzoyl cyanide, 2,5-dichlorobenzoyl cyanide, 2,6-dichlorobenzoyl cyanide, 3,4-dichlorobenzoyl cyanide or 2-chlorobenzoyl cyanide or an isomer thereof.

7. Process according to claim 1, wherein the benzoyl chloride of the formula (II) is reacted with 1.0 to 1.3 molar equivalents of copper cyanide.

8. Process according to claim 1, wherein the benzoyl chloride of the formula (II) is reacted with 1.0 to 1.1 molar equivalents of copper cyanide.

9. Process according to claim 1, wherein the reaction temperature is between 155 and 160° C.

10. Process according to claim 1, wherein the reaction time is not more than 4 hours.

11. Process according to claim 1, wherein in that the substituted benzoyl cyanides are obtained with a purity greater than 94%, and simultaneously comprise not more than 2% of the corresponding acid secondary component and not more than 6% of the corresponding anhydride components.

12. Process according to claim 1, wherein the substituted benzoyl cyanides are obtained with a purity greater than 95%, and simultaneously comprise not more than 1% of the corresponding acid secondary component and not more than 4% of the corresponding anhydride components.

13. Process according to claim 1, wherein the substituted benzoyl cyanides are obtained with a purity greater than 96%, and simultaneously comprise not more than 0.6% of the corresponding acid secondary component and not more than 3% of the corresponding anhydride components.

14. Substituted benzoyl cyanide obtainable claim 1.
